(19) United States
(12) Patent Application Publication
(10) Pub. No.: US 2022/0249762 A1
(43) Pub. Date: Aug. 11, 2022

(54) Composite Dressing For Tissue Closure With Negative Pressure

(71) Applicant: KCI Licensing, Inc., San Antonio, TX (US)

(72) Inventors: Christopher Brian LOCKE, Bournemouth (GB); Benjamin Andrew PRATT, Poole (GB)

(21) Appl. No.: 17/596,373

(22) PCT Filed: May 5, 2020
(86) PCT No.: PCT/US2020/031465

§ 371 Date: Dec. 8, 2021

Related U.S. Application Data
(60) Provisional application No. 62/860,735, filed on Jun. 12, 2019.

(51) Int. Cl. A61M 1/00

(52) U.S. Cl. CPC A61M 1/915

(57) ABSTRACT

A dressing or a tissue interface for treating a tissue site with negative pressure may comprise a fluid control layer, a base manifold, and a closure manifold layer. The fluid control layer may comprise a plurality of fluid restrictions, and the base manifold may be disposed adjacent to the fluid restrictions. The closure manifold may have perforations adjacent to the base manifold layer. Additionally, the base manifold layer may have a first density, and the closure manifold layer may have a second density, wherein the second density is less than the first density. The closure manifold may be configured to deform laterally at a second negative pressure that is less than the first negative pressure.

### RELATED APPLICATIONS

The present application claims priority to U.S. Provisional Patent Application No. 62/860,735, entitled “Collapsible, Customizable, Wound Filler, Incorporating Fenestrated Interface Layer,” filed Jun. 12, 2019, which is incorporated herein by reference for all purposes.

### TECHNICAL FIELD

The invention set forth in the appended claims relates generally to tissue treatment systems and more particularly, but without limitation, to systems, apparatuses, and methods for treating tissue with negative pressure.

### BACKGROUND

Clinical studies and practice have shown that reducing pressure in proximity to a tissue site can augment and accelerate growth of new tissue at the tissue site. The applications of this phenomenon are numerous, but reduced pressure has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

There is also widespread acceptance that cleansing a tissue site can be highly beneficial for new tissue growth. For example, a wound or a cavity can be washed out with a liquid solution for therapeutic purposes. These practices are commonly referred to as “irrigation” and “lavage” respectively. “Instillation” is another practice that generally refers to a process of slowly introducing fluid to a tissue site and leaving the fluid for a prescribed period of time before removing the fluid. For example, instillation of topical treatment solutions over a wound bed can be combined with negative-pressure therapy to further promote wound healing by loosening soluble contaminants in a wound bed and removing infectious material. As a result, soluble bacterial burden can be decreased, contaminants removed, and the wound cleansed.

While the clinical benefits of negative-pressure therapy and/or instillation therapy are widely known, improvements to therapy systems, components, and processes may benefit healthcare providers and patients.

### BRIEF SUMMARY

New and useful systems, apparatuses, and methods for treating tissue in a negative-pressure therapy environment are set forth in the appended claims. Illustrative embodiments are also provided to enable a person skilled in the art to make and use the claimed subject matter.

For example, in some embodiments, a tissue interface for treating a tissue site may comprise three functional layers, including a fenestrated film layer, a flexible manifold base layer, and a collapsible manifold layer. In more particular examples, the film layer may be an adhesive-backed polymer film layer, which can be fenestrated. The manifold base may be attached to the film layer. For example, the manifold base may be a thin foam layer bonded to the film layer using the adhesive backing. Additionally, or alternatively, the foam layer may be flame-laminated to or co-extruded with the film layer in some embodiments. A suitable foam layer may be a felted, reticulated foam material, which can be skived or otherwise cut down. The conformability and flexibility of the material may be controlled and defined by the initial felting in conjunction with the skiving thickness, which can be modulated. The manifold base layer may be adhered to the collapsible manifold layer, which may comprise a larger, perforated and sectioned foam element. In some examples, the collapsible manifold layer may be bonded or flame-laminated to the manifold base layer. The collapsible manifold layer may also provide a primary means for delivering lateral and radial collapse under negative pressure. In some embodiments, the collapsible manifold layer may be a non-felted, reticulated foam. In other embodiments, the collapsible manifold layer may be a felted, reticulated foam, which can allow for greater perforation area and modulus stiffness. Perforations in such embodiments may provide a primary means for fluid flow, instead of or in addition to the cellular structure of the foam. The base manifold layer should be sufficiently structural to hold itself against the initial collapse of the collapsible manifold layer, without preventing lateral contraction.

In some embodiments, the collapsible manifold layer may have a pattern of holes configured to increase closure forces. The holes may be arranged normally over the collapsible manifold layer or may be aligned and have shapes similar to the shape of the collapsible manifold layer.

In some embodiments, the tissue interface may additionally have a silicone layer with perforations, which can at least partially align with the fenestrations in the film layer. Additionally, some embodiments of the tissue interface may be incorporated into a dressing configured to treat tissue with negative pressure.

More generally, a dressing or a tissue interface for treating a tissue site with negative pressure may comprise a fluid control layer, a first manifold layer, and a second manifold layer. The fluid control layer may comprise a plurality of fluid restrictions, and the first manifold layer may be disposed adjacent to the fluid restrictions. The second manifold layer may have perforations adjacent to the first manifold layer. Additionally, the first manifold layer may have a first density, and the second manifold layer may have a second density, wherein the second density is less than the first density. A suitable ratio of the first density to the second density may be in a range of about 2.5 to about 3.3. For example, the first density may be about 0.65 grams per cubic centimeter, and the second density may be about 0.2 to about 0.26 grams per cubic centimeter.

In more particular examples, the perforations of the second manifold layer may define an open area of about 30% to about 70%. In some embodiments, the perforations may be arranged in a uniform pattern, and the perforations may be separated by struts having a substantially uniform thickness.

Alternatively, other example embodiments may comprise a first layer, a second layer and a third layer. The first layer may comprise or consist essentially of a fluid control layer having a plurality of fluid restrictions. The second layer may comprise or consist of a base manifold disposed adjacent to the fluid restrictions and may be configured to deform laterally at a first negative pressure. The third layer may comprise or consist of a closure manifold disposed adjacent to the base manifold. The closure manifold may be configured to deform laterally at a second negative pressure that is less than the first negative pressure. In some examples, the first negative pressure may be at least 60 mmHg, and the second negative pressure may be less than 50 mmHg.

In some embodiments, the dressing or tissue interface may be used to treat a tissue site with negative pressure. For example, a method for treating a tissue site with negative pressure may comprise applying the tissue interface to the tissue site, attaching a cover to an attachment surface around the tissue site to seal the tissue interface over the tissue site, fluidly coupling the tissue interface to a negative-pressure source, and applying negative pressure from the negative-pressure source to the tissue interface, which can promote closure and granulation of the tissue site.

Some embodiments can provide a manifold structure that presents a substantially even surface topology to a wound, and can reduce size and area of a wound by laterally collapsing under negative pressure. Some embodiments may also prevent growth of granulation tissue into the tissue interface, which can substantially reduce trauma on removal.

Other objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment and instillation treatment in accordance with this specification;

FIG. 2 is an assembly view of an example of a tissue interface that may be associated with some embodiments of the therapy system of FIG. 1;

FIG. 3 is a top view of the tissue interface of FIG. 2, as assembled;

FIG. 4 is a side view of the tissue interface of FIG. 3;

FIG. 5 is a bottom view of the tissue interface of FIG. 3;

FIG. 6 is an assembly view of another example of a tissue interface;

FIG. 7 is a bottom view of the tissue interface of FIG. 6, as assembled;

FIG. 8 is an assembly view of another example of a tissue interface;

FIG. 9 is a bottom view of the tissue interface of FIG. 8, as assembled;

FIG. 10 is an assembly view of an example of a dressing with the tissue interface of FIG. 6;

FIG. 11 is a top view of the dressing in the example of FIG. 10, as assembled;

FIG. 12 is an assembly view of an example of a dressing with the tissue interface of FIG. 8;

FIG. 13 is a top view of the dressing of FIG. 12; and

FIG. 14 is a schematic diagram of an example of a dressing applied to a tissue site.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

Exemplary Therapy System

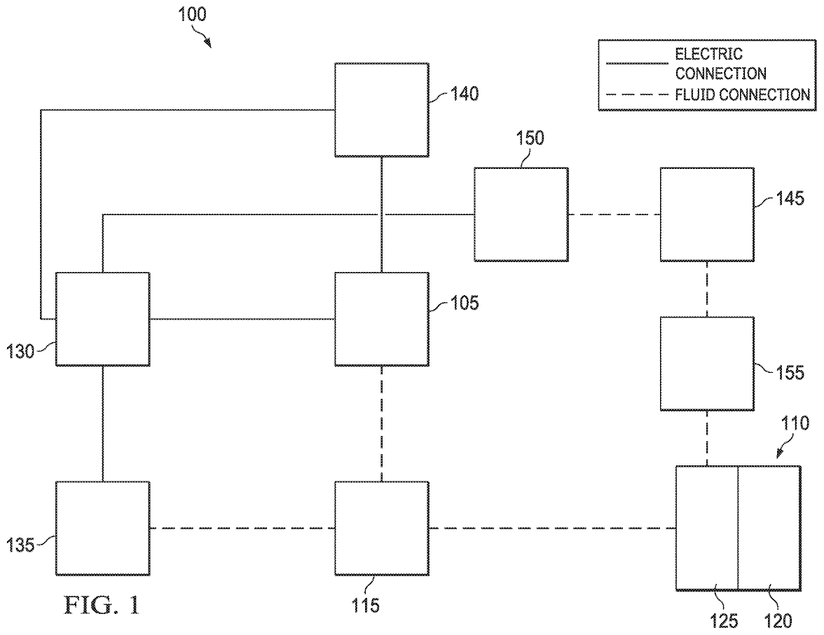

FIG. 1 is a simplified functional block diagram of an example embodiment of a therapy system 100 that can provide negative-pressure therapy with instillation of topical treatment solutions to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Tex.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as fluid from a source of instillation solution, across a tissue site.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, TEGADERM drape material, commercially available from 3M Company, Minneapolis Minn.; polyurethane (PU) drape material, commercially available from Avery Dennison Corporation, Pasadena, Calif.; polyether block polyamide copolymer (PEBAX), for example, from Arkema S.A., Colombes, France; and INSPIRE 2301 and INSPIRE 2327 polyurethane films, commercially available from Expopack Advanced Coatings, Wrexham, United Kingdom. In some embodiments, the cover 125 may comprise INSPIRE 2301 matte polyurethane film having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

In some embodiments, the controller 130 may receive and process data from one or more sensors, such as the first sensor 135 and the second sensor 140. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

The therapy system 100 may also include a source of instillation solution. For example, a solution source 145 may be fluidly coupled to the dressing 110, as illustrated in the example embodiment of FIG. 1. The solution source 145 may be fluidly coupled to a positive-pressure source such as a positive-pressure source 150, a negative-pressure source such as the negative-pressure source 105, or both in some embodiments. A regulator, such as an instillation regulator 155, may also be fluidly coupled to the solution source 145 and the dressing 110 to ensure proper dosage of instillation solution (e.g. saline) to a tissue site. For example, the instillation regulator 155 may comprise a piston that can be pneumatically actuated by the negative-pressure source 105 to draw instillation solution from the solution source during a negative-pressure interval and to instill the solution to a dressing during a venting interval. Additionally or alternatively, the controller 130 may be coupled to the negative-pressure source 105, the positive-pressure source 150, or both, to control dosage of instillation solution to a tissue site. In some embodiments, the instillation regulator 155 may also be fluidly coupled to the negative-pressure source 105 through the dressing 110, as illustrated in the example of FIG. 1.

The solution source 145 may be representative of a container, canister, pouch, bag, or other storage component, which can provide a solution for instillation therapy. Compositions of solutions may vary according to a prescribed therapy, but examples of solutions that may be suitable for some prescriptions include hypochlorite-based solutions, silver nitrate (0.5%), sulfur-based solutions, biguanides, cationic solutions, and isotonic solutions.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130, the solution source 145, and other components into a therapy unit.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

The dressing 110 can provide a sealed therapeutic environment proximate to a tissue site, substantially isolated from the external environment, and the negative-pressure source 105 can reduce the pressure in the sealed therapeutic environment. Negative pressure applied through the tissue interface 120 in the sealed therapeutic environment can induce macro-strain and micro-strain in a tissue site. Negative pressure can also remove exudate and other fluid from a tissue site, which can be collected in container 115.

Exemplary Tissue Interface Configurations

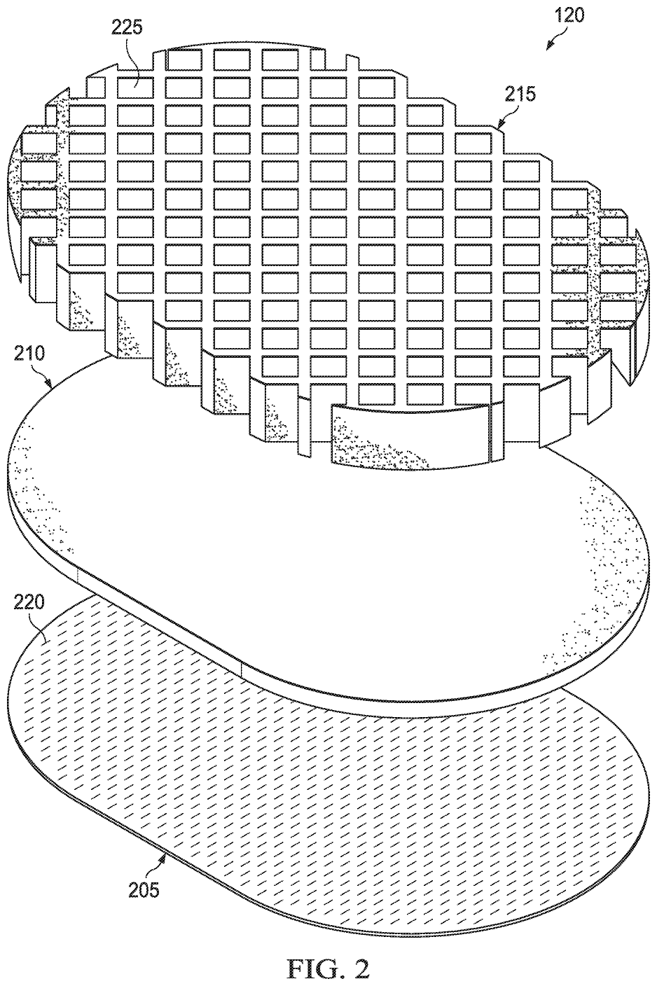

FIG. 2 is an assembly view of an example of the tissue interface 120 of FIG. 1, illustrating additional details that may be associated with some embodiments. As illustrated in the example of FIG. 2, some embodiments of the tissue interface 120 may have more than one layer. The tissue interface 120 of FIG. 2 comprises a first layer 205, a second layer 210, and a third layer 215.

The first layer 205 may comprise or consist essentially of a means for controlling or managing fluid flow. In some embodiments, the first layer 205 may be a fluid control layer comprising or consisting essentially of a liquid-impermeable, elastomeric material. For example, the first layer 205 may comprise or consist essentially of a polymer film, such as a polyurethane film, having an MVTR (upright cup technique) of about 2600 g/m²/24 hours and a thickness of about 30 microns. In some embodiments, the first layer 205 may comprise or consist essentially of the same material as the cover 125. The first layer 205 may also have a smooth or matte surface texture in some embodiments. A glossy or shiny finish that is equal to a grade B3 or finer according to the SPI (Society of the Plastics Industry) standards may be particularly advantageous for some applications. In some embodiments, variations in surface height may be limited to acceptable tolerances. For example, the surface of the first layer 205 may have a substantially flat surface, with height variations limited to 0.2 millimeters over a centimeter.

In some embodiments, the first layer 205 may be hydrophobic. The hydrophobicity of the first layer 205 may vary, but the first layer 205 may have a contact angle with water of at least ninety degrees in some embodiments. In some embodiments the first layer 205 may have a contact angle with water of no more than 150 degrees. For example, in some embodiments, the contact angle of the first layer 205 may be in a range of at least 90 degrees to about 120 degrees, or in a range of at least 120 degrees to 150 degrees.

Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTA125, FTA200, FTA2000, and FTA4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles herein represent averages of 5-9 measured values, discarding both the highest and lowest measured values.

The hydrophobicity of the first layer 205 may be further enhanced with a hydrophobic coating of other materials, such as silicones and fluorocarbons, either as coated from a liquid, or plasma coated.

The first layer 205 may also be suitable for welding to other layers, including the second layer 210. For example, the first layer 205 may be adapted for welding to polyurethane foams using heat, radio frequency (RF) welding, or other methods to generate heat such as ultrasonic welding. RF welding may be particularly suitable for more polar materials, such as polyurethane, polyamides, polyesters and acrylates. Sacrificial polar interfaces may be used to facilitate RF welding of less polar film materials, such as polyethylene.

The area density of the first layer 205 may vary according to a prescribed therapy or application. In some embodiments, an area density of less than 40 grams per square meter may be suitable, and an area density of about 20-30 grams per square meter may be particularly advantageous for some applications.

In some embodiments, for example, the first layer 205 may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. Films may be clear, colored, or printed. More polar films suitable for laminating to a polyethylene film include polyamide, co-polyesters, ionomers, and acrylics. To aid in the bond between a polyethylene and polar film, tie layers may be used, such as ethylene vinyl acetate, or modified polyurethanes. An ethyl methyl acrylate (EMA) film may also have suitable hydrophobic and welding properties for some configurations.

As illustrated in the example of FIG. 2, the first layer 205 may have one or more fluid restrictions 220, which can be distributed uniformly or randomly across the first layer 205. The fluid restrictions 220 may be bi-directional and pressure-responsive. For example, each of the fluid restrictions 220 generally may comprise or consist essentially of an elastic passage that is normally unstrained to substantially reduce liquid flow, and can expand or open in response to a pressure gradient. In some embodiments, the fluid restrictions 220 may comprise or consist essentially of perforations in the first layer 205. Perforations may be formed by removing material from the first layer 205. For example, perforations may be formed by cutting through the first layer 205, which may also deform the edges of the perforations in some embodiments. In the absence of a pressure gradient across the perforations, the passages may be sufficiently small to form a seal or fluid restriction, which can substantially reduce or prevent liquid flow. Additionally, or alternatively, one or more of the fluid restrictions 220 may be an elastomeric valve that is normally closed when unstrained to substantially prevent liquid flow, and can open in response to a pressure gradient. A fenestration in the first layer 205 may be a suitable valve for some applications. Fenestrations may also be formed by removing material from the first layer 205, but the amount of material removed and the resulting dimensions of the fenestrations may be up to an order of magnitude less than perforations, and may not deform the edges.

The second layer 210 generally comprises or consists essentially of a base manifold or a manifold layer, which provides a means for collecting or distributing fluid across the tissue interface 120 under pressure. For example, the second layer 210 may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid, such as from a source of instillation solution, across the tissue interface 120.

In some illustrative embodiments, the pathways of the second layer 210 may be interconnected to improve distribution or collection of fluids. In some illustrative embodiments, the second layer 210 may comprise or consist essentially of a porous material having interconnected fluid pathways. Examples of suitable porous material that comprise or can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, the second layer 210 may additionally or alternatively comprise projections that form interconnected fluid pathways. For example, the second layer 210 may be molded to provide surface projections that define interconnected fluid pathways.

In some embodiments, the second layer 210 may comprise or consist essentially of a reticulated foam having pore sizes and free volume that may vary according to needs of a prescribed therapy. For example, a reticulated foam of polyvinyl alcohol having a density of about 0.06 to 0.7 grams per cubic centimeter, a minimum compression stress of about 5000 Pa, and pore sizes in a range of about 0.7 millimeters to about 2 millimeters may be particularly suitable for some configurations. More generally, a reticulated foam having a free volume of at least 90% may be suitable for many therapy applications, and a foam having an average pore size in a range of 400-600 microns may be particularly suitable for some types of therapy. The tensile strength of the second layer 210 may also vary according to needs of a prescribed therapy. For example, the tensile strength of a foam may be increased for instillation of topical treatment solutions. The 25% compression load deflection of the second layer 210 may be at least 0.35 pounds per square inch, and the 65% compression load deflection may be at least 0.43 pounds per square inch. In some embodiments, the tensile strength of the second layer 210 may be at least 10 pounds per square inch. The second layer 210 may have a tear strength of at least 2.5 pounds per inch. In some embodiments, the second layer 210 may be a foam comprised of polyols such as polyester or polyether, isocyanate such as toluene diisocyanate, and polymerization modifiers such as amines and tin compounds. In some examples, the second layer 210 may be a reticulated polyurethane foam such as used in GRANUFOAM™ dressing or V.A.C. VERAFLO™ dressing, both available from KCI of San Antonio, Tex.

Other suitable materials for the second layer 210 may include non-woven fabrics (Libeltex, Freudenberg), three-dimensional (3D) polymeric structures (molded polymers, embossed and formed films, and fusion-bonded films [Supracor]), and mesh, for example.

In some examples, the second layer 210 may include a 3D textile, such as various textiles commercially available from Baltex, Muller, and Heathcoates. A 3D textile of polyester fibers may be particularly advantageous for some embodiments. For example, the second layer 210 may comprise or consist essentially of a three-dimensional weave of polyester fibers. In some embodiments, the fibers may be elastic in at least two dimensions. A puncture-resistant fabric of polyester and cotton fibers having a weight of about 650 grams per square meter and a thickness of about 1-2 millimeters may be particularly advantageous for some embodiments. Such a puncture-resistant fabric may have a warp tensile strength of about 330-340 kilograms and a weft tensile strength of about 270-280 kilograms in some embodiments. Another particularly suitable material may be a polyester spacer fabric having a weight of about 470 grams per square meter, which may have a thickness of about 4-5 millimeters in some embodiments. Such a spacer fabric may have a compression strength of about 20-25 kilopascals (at 40% compression). Additionally, or alternatively, the second layer 210 may comprise or consist of a material having substantial linear stretch properties, such as a polyester spacer fabric having 2-way stretch and a weight of about 380 grams per square meter. A suitable spacer fabric may have a thickness of about 3-4 millimeters, and may have a warp and weft tensile strength of about 30-40 kilograms in some embodiments. The fabric may have a close-woven layer of polyester on one or more opposing faces in some examples. In some embodiments, a woven layer may be advantageously disposed on a first layer 205 to face a tissue site.

The third layer 215 may comprise or consist essentially of a closure manifold or manifold layer. In some embodiments, the third layer 215 may have material properties that are the same or similar to the second layer 210. For example, the third layer 215 may comprise a reticulated foam having a density in a range of about 0.2 to about 0.3 grams per cubic centimeter, a free volume of at least 90%, and an average pore size in a range of 400-600 microns. The foam may be felted to increase modulus stiffness in some embodiments.

Additionally, the third layer 215 may have a plurality of perforations, such as holes 225, as illustrated in the example of FIG. 2.

Individual components of the tissue interface 120 may be bonded or otherwise secured to one another with a solvent or non-solvent adhesive, or with thermal welding, for example, without adversely affecting fluid management. Hot-melt adhesives or other suitable adhesives may be selected to maintain a more permanent bond or may be designed to preferentially release the third layer 215 to allow replacement.

The first layer 205, the second layer 210, the third layer 215, or various combinations may be assembled before application or in situ. For example, the second layer 210 may be laminated to the first layer 205 in some embodiments. In some embodiments, one or more layers of the tissue interface 120 may be coextensive. For example, the second layer 210 may be cut flush with the edge of the third layer 215. In some embodiments, the tissue interface 120 may be provided as composite article. For example, the third layer 215 may be coupled to the second layer 210 and the second layer 210 may be coupled to the first layer 205, wherein the first layer 205 may be configured to face a tissue site.

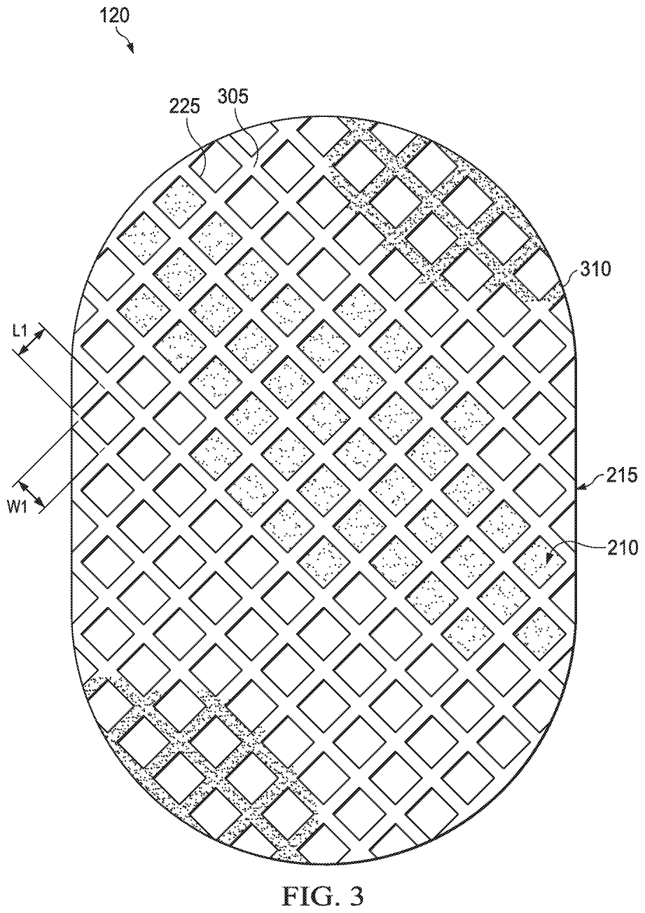

FIG. 3 is a top view of the tissue interface 120 of FIG. 2, as assembled, illustrating additional details that may be associated with some embodiments. For example, the holes 225 may be through-holes, as illustrated in FIG. 3, which can be separated by a web of struts 305. The struts 305 in the example of FIG. 3 have a substantially uniform thickness. The holes 225 may additionally be characterized by various properties, such as shape, size, pattern, and orientation of the pattern.

For example, the shape of the holes 225 may be characterized as open right cylinders. The right section of the holes 225 in FIG. 3 are square. More generally, the right section of the holes 225 may be a polygon, and may be a regular polygon such as a triangle, a rectangle, or pentagon. Other suitable shapes may include circles, stars, ovals, or a combination of shapes, and the struts 305 may not have a uniform thickness. Additionally, the third layer 215 may be partially cut between the holes 225 to increase flexibility of the third layer 215.

The size of the holes 225 may be specified by a length L1 (the longer of two dimensions) and width W1 (the shorter of two dimensions) in some examples. In some embodiments, each of the holes may have substantially the same width W1 and length L1, as illustrated in the example of FIG. 3, and the size of the holes 225 may be specified by a single dimension, such as the width W1. A width W1 and a length L1 of about 5 millimeters to about 20 millimeters may be suitable for some embodiments. Each of the holes 225 may have uniform or similar sizes. For example, in some embodiments, each of the holes 225 may have substantially the same width W1, as illustrated in the example of FIG. 3. In other embodiments, geometric properties of the holes 225 may vary. For example, the width of the holes 225 may vary depending on the position of the holes 225 in the third layer 215. In some embodiments, the width of the holes 225 may be larger in a peripheral area than an interior area of the third layer 215. At least some of the holes 225 may be positioned on one or more edges 310 of the third layer 215, and may have an interior cut open or exposed at one or more of the edges 310.

In some examples, the holes 225 may be arranged in a uniform pattern. For example, the holes 225 may have a uniform distribution pattern, such as an arrangement of rows. In other examples, the holes 225 may be randomly distributed in the third layer 215. The holes 225 may be arranged with no alignment to the shape of the third layer 215 in some embodiments.

The third layer 215 may also be characterized by an open area, which can be formed by the holes 225. The open area may be expressed as a percentage of an area defined by edges 310 of the third layer 215. An open area of about 30 percent to about 70 percent of the area of the third layer 215 may be suitable for some examples.

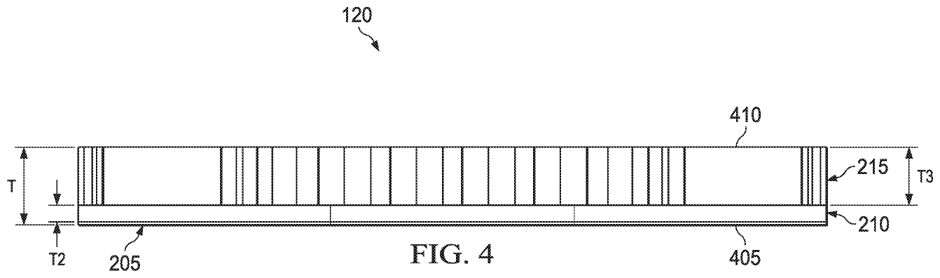

FIG. 4 is a side view of the tissue interface 120 of FIG. 3, illustrating additional details that may be associated with some examples. For example, as illustrated in FIG. 3, the first layer 205, the second layer 210, and the third layer 215 may be assembled in a stacked relationship so that the second layer 210 is disposed between the first layer 205 and the third layer 215. The second layer 210 may provide substantially continuous and even surfaces adjacent to the first layer 205 and the third layer 215. The tissue interface 120 generally has a first planar surface 405 and a second planar surface 410 opposite the first planar surface 405. In FIG. 4, the first planar surface 405 is defined by a surface of the first layer 205, and the second planar surface 410 is defined by a surface of the third layer 215.

The thickness T of the tissue interface 120, and each of the layers, between the first planar surface 405 and the second planar surface 410 may also vary according to needs of a prescribed therapy. For example, the thickness T2 of the second layer 210 may be decreased to relieve stress on other layers and to reduce tension on peripheral tissue. The thickness T2 of the second layer 210 can also affect the conformability of the second layer 210. In some embodiments, a suitable reticulated foam may have a thickness T2 in a range of about 3 millimeters to 6 millimeters, and about 1 millimeter to about 3 millimeter if felted. Fabrics, including suitable 3D textiles and spacer fabrics, may have a thickness T2 in a range of about 2 millimeters to about 8 millimeters. A suitable reticulated foam may have a thickness T3 in a range of about 10 millimeters to about 20 millimeters, and may be about 6 millimeters to about 10 millimeters if felted.

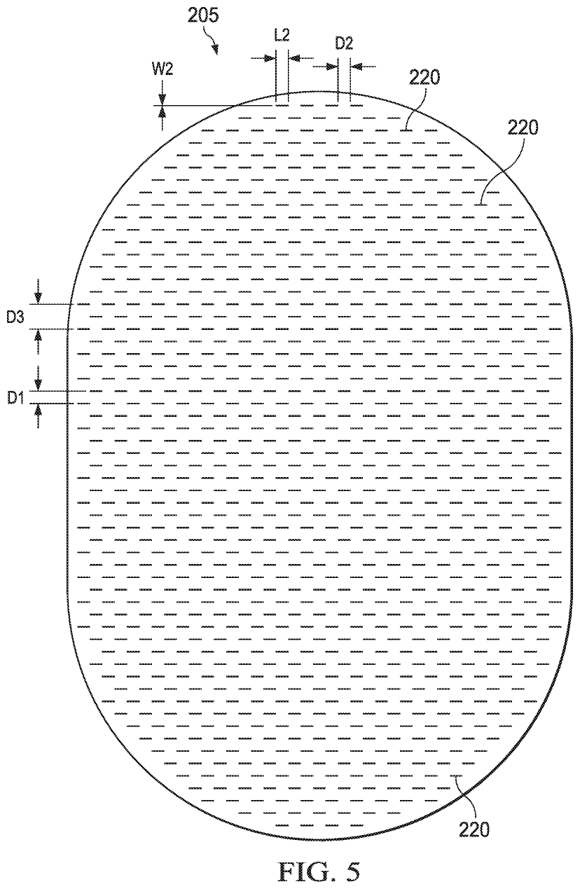

FIG. 5 is a bottom view of the tissue interface 120 of FIG. 3, illustrating additional details that may be associated with some embodiments. For example, as illustrated in the example of FIG. 5, some embodiments of the fluid restrictions 220 may comprise or consist essentially of one or more slits, slots or combinations of slits and slots in the first layer 205. In some examples, the fluid restrictions 220 may comprise or consist of linear slots, which can be characterized by a length L2 and a width W2. A length L2 of at least 2 millimeters and not greater than about 4 millimeters may be suitable for some embodiments. A width W2 of less than 1 millimeter may also be suitable for some embodiments. A length L2 of about 3 millimeters and a width W2 of about 0.5 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

FIG. 5 additionally illustrates an example of a uniform distribution pattern of the fluid restrictions 220. In FIG. 5, the fluid restrictions 220 are substantially coextensive with the first layer 205, and are distributed across the first layer 205 in a grid of parallel rows and columns, in which the slots are also mutually parallel to each other. In some embodiments, the rows may be spaced a distance D1. A distance D1 of about 3 millimeters on center may be suitable for some embodiments. The fluid restrictions 220 within each of the rows may be spaced a distance D2, which may be about 3 millimeters on center in some examples. The fluid restrictions 220 in adjacent rows may be aligned or offset in some embodiments. For example, adjacent rows may be offset, as illustrated in FIG. 5, so that the fluid restrictions 220 are aligned in alternating rows and separated by a distance D3, which may be about 6 millimeters in some embodiments. The spacing of the fluid restrictions 220 may vary in some embodiments to increase the density of the fluid restrictions 220 according to therapeutic requirements.

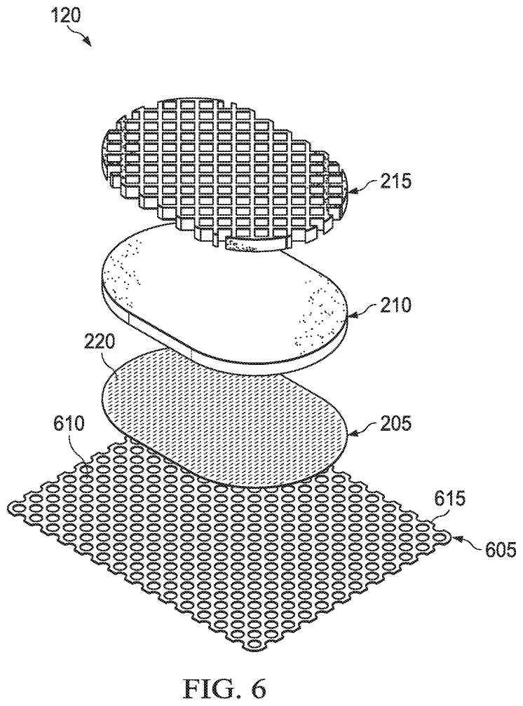

FIG. 6 is an assembly view of another example of the tissue interface 120, illustrating additional details that may be associated with some examples. For example, the tissue interface 120 of FIG. 6 further includes a fourth layer 605. The fourth layer 605 may comprise or consist essentially of a sealing layer formed from a soft, pliable material suitable for providing a fluid seal with a tissue site, such as a suitable gel material, and may have a substantially flat surface. For example, the fourth layer 605 may comprise, without limitation, a silicone gel, a soft silicone, hydrocolloid, hydrogel, polyurethane gel, polyolefin gel, hydrogenated styrenic copolymer gel, a foamed gel, a soft closed cell foam such as polyurethanes and polyolefins coated with an adhesive, polyurethane, polyolefin, or hydrogenated styrenic copolymers. In some embodiments, the fourth layer 605 may have a thickness between about 200 microns (μm) and about 1000 microns (μm). In some embodiments, the fourth layer 605 may have a hardness between about 5 Shore 00 and about 80 Shore 00. Further, the fourth layer 605 may be comprised of hydrophobic or hydrophilic materials.

In some embodiments, the fourth layer 605 may be a hydrophobic-coated material. For example, the fourth layer 605 may be formed by coating a spaced material, such as, for example, woven, nonwoven, molded, or extruded mesh with a hydrophobic material. The hydrophobic material for the coating may be a soft silicone, for example.

The fourth layer 605 may have a plurality of apertures 610. The apertures 610 may be formed by cutting, perforating, or by application of local RF or ultrasonic energy, for example, or by other suitable techniques for forming an opening or perforation in the fourth layer 605. The apertures 610 may have a uniform distribution pattern, or may be randomly distributed across the fourth layer 605. The apertures 610 in the fourth layer 605 may have many shapes, including circles, squares, diamonds, stars, ovals, polygons, slits, complex curves, rectilinear shapes, triangles, for example, or may have some combination of such shapes.

Each of the apertures 610 may have uniform or similar geometric properties. For example, in some embodiments, each of the apertures 610 may be circular apertures, having substantially the same diameter. In some embodiments, each of the apertures 610 may have a diameter of about 1 millimeter to about 50 millimeters. In other embodiments, the diameter of each of the apertures 610 may be about 1 millimeter to about 20 millimeters.

In other embodiments, geometric properties of the apertures 610 may vary. For example, the diameter of the apertures 610 may vary depending on the position of the apertures 610 in the fourth layer 605. At least one of the apertures 610 may be positioned at edges 615 of the fourth layer 605, and may have an interior cut open or exposed at the edges 615. The apertures 610 positioned proximate to or at the edges 615 may be spaced substantially equidistant around the edges 615, as shown in the example of FIG. 6. Alternatively, the spacing of the apertures 610 proximate to or at the edges 630 may be irregular.

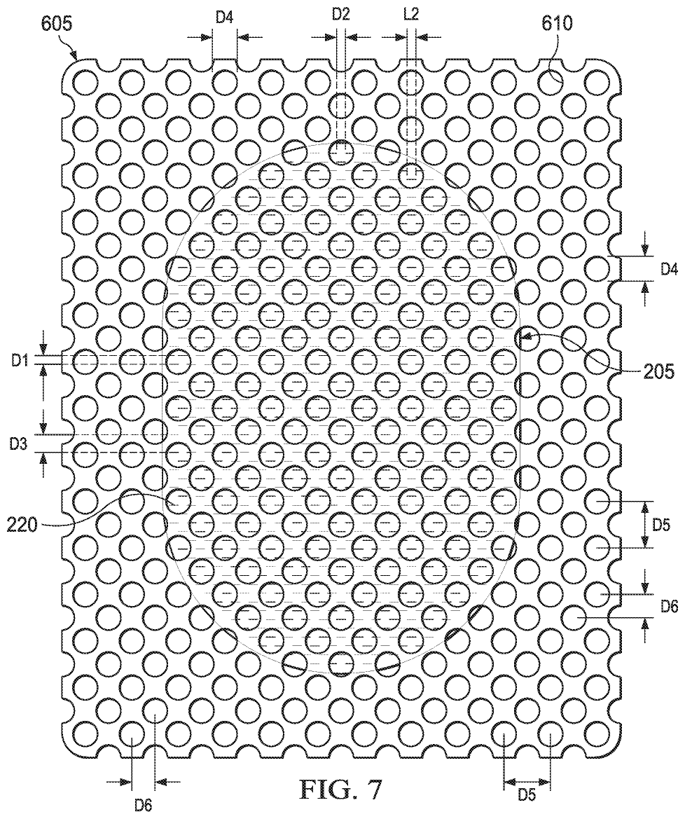

FIG. 7 is a bottom view of the tissue interface 120 of FIG. 6, as assembled, illustrating additional details that may be associated with some embodiments. In the example of FIG. 7, the apertures 610 are generally circular and have a diameter D4, which may be about 6 millimeters to about 8 millimeters in some embodiments. A diameter D4 of about 7 millimeters may be particularly suitable for some embodiments. FIG. 7 also illustrates an example of a uniform distribution pattern of the apertures 610. In FIG. 7, the apertures 610 are distributed across the fourth layer 605 in a grid of parallel rows and columns. Within each row and column, the apertures 605 may be equidistant from each other, as illustrated in the example of FIG. 7. FIG. 7 illustrates one example configuration that may be particularly suitable for many applications, in which the apertures 610 are spaced a distance D5 apart along each row and column, with an offset of D6. In some examples, the distance D5 may be about 9 millimeters to about 10 millimeters, and the offset D6 may be about 8 millimeters to about 9 millimeters.

As illustrated in FIG. 7, more than one of the fluid restrictions 220 may be aligned, overlapping, in registration with, or otherwise fluidly coupled to the apertures 610 in some embodiments. In some embodiments, one or more of the fluid restrictions 220 may be only partially registered with the apertures 610. The apertures 610 in the example of FIG. 7 are generally sized and configured so that at least four of the fluid restrictions 220 are registered with each one of the apertures 610. In other examples, one or more of the fluid restrictions 220 may be registered with more than one of the apertures 610. For example, any one or more of the fluid restrictions 220 may be a perforation or a fenestration that extends across two or more of the apertures 610. Additionally or alternatively, one or more of the fluid restrictions 220 may not be registered with any of the apertures 610.

As illustrated in the example of FIG. 7, the apertures 610 may be sized to expose a portion of the first layer 205, the fluid restrictions 220, or both through the fourth layer 605. The apertures 610 in the example of FIG. 7 are generally sized to expose more than one of the fluid restrictions 220. Some or all of the apertures 610 may be sized to expose two or three of the fluid restrictions 220. In some examples, the length L of each of the fluid restrictions 220 may be substantially smaller than the diameter of each of the apertures 610. More generally, the average dimensions of the fluid restrictions 220 are substantially smaller than the average dimensions of the apertures 610. In some examples, the apertures 610 may be elliptical, and the length of each of the fluid restrictions 220 may be substantially smaller than the major axis or the minor axis. In some embodiments, though, the dimensions of the fluid restrictions 220 may exceed the dimensions of the apertures 610, and the size of the apertures 610 may limit the exposure of the fluid restrictions 220.

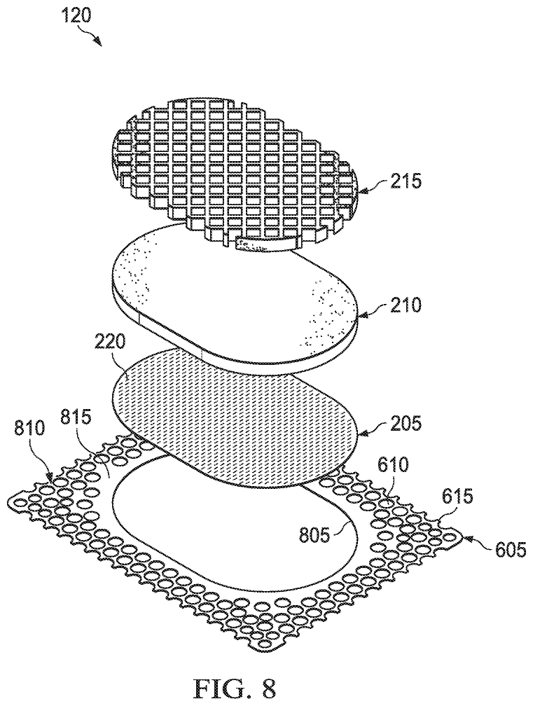

FIG. 8 is an assembly view of another example of the tissue interface 120, illustrating additional details that may be associated with some embodiments. For example, some embodiments of the fourth layer 605 may have a treatment aperture 805. The apertures 610 may be disposed in a periphery 810 around the treatment aperture 805.

The fourth layer 605 may have an interior border 815 around the treatment aperture 805, which may be substantially free of the apertures 610, as illustrated in the example of FIG. 8. In some examples, as illustrated in FIG. 8, the treatment aperture 805 may be symmetrical and centrally disposed in the fourth layer 605, forming an open central window.

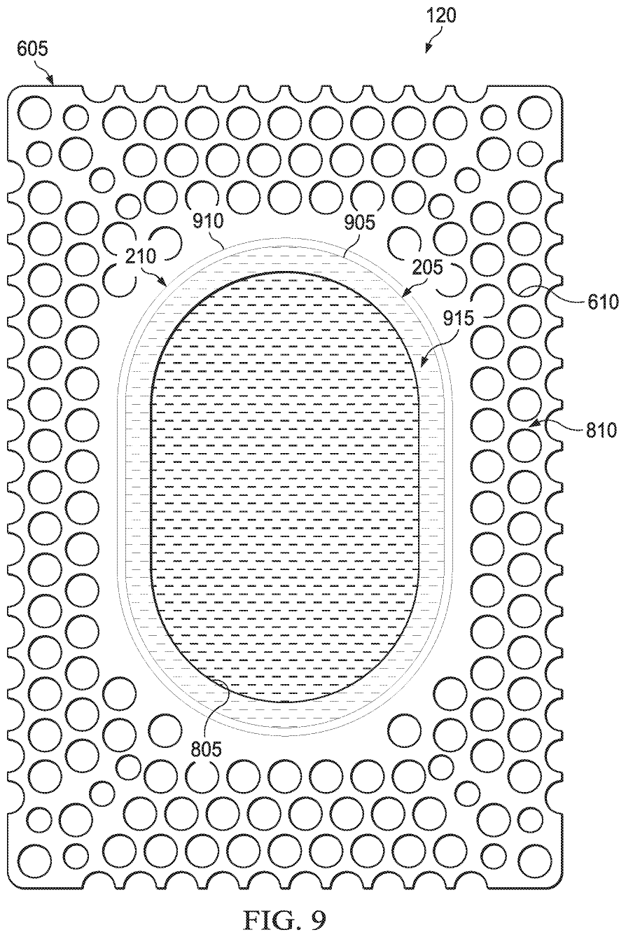

FIG. 9 is a bottom view of the tissue interface 120 of FIG. 8, as assembled, illustrating additional details that may be associated with some embodiments. As illustrated in the example of FIG. 9, the first layer 205 may be disposed over the treatment aperture 805. A substantial number of the fluid restrictions 220 may be aligned or otherwise exposed through the treatment aperture 805, and at least some portion of the second layer 210 may be in fluid communication with the fluid restrictions 220. In some embodiments, the first layer 205 and the second layer 210 may be substantially aligned with the treatment aperture 805, or may extend across the treatment aperture 805. The treatment aperture 805 may be complementary or correspond to a surface area of the first layer 205 in some examples. For example, the treatment aperture 805 may form a frame, window, or other opening around a surface of the first layer 205.

In some embodiments, the apertures 610 disposed in the periphery 810 may have a diameter between about 5 millimeters and about 10 millimeters. A range of about 7 millimeters to about 9 millimeters may be suitable for some examples. In some embodiments, the apertures 610 disposed in the corners may have a diameter between about 7 millimeters and about 8 millimeters.

Additionally, the first layer 205 may have a first edge 905, and the second layer 210 may have a second edge 910. In some examples, the first edge 905 and the second edge 910 may have substantially the same shape so that adjacent faces of the first layer 205 and the second layer 210 are geometrically similar. The first edge 905 and the second edge 910 may also be congruent in some examples, so that adjacent faces of the first layer 205 and the second layer 210 are substantially coextensive and have substantially the same surface area. In the example of FIG. 9, the first edge 905 of the first layer 205 defines a smaller face than the face defined by the second edge 910 of the second layer 210, and the larger face of the second layer 210 can extend past the smaller face of the first layer 205. The third layer 215 (not visible) may also have a geometrically similar shape as the first layer 205, the second layer 210, or both.

The faces defined by the first edge 905, the second edge 910, or both may also be geometrically similar to the treatment aperture 805 in some embodiments, as illustrated in the example of FIG. 9, and may be larger than the treatment aperture 805. The fourth layer 605 may have an overlay margin 915 around the treatment aperture 805, which may have an additional adhesive disposed therein. As illustrated in the example of FIG. 9, the treatment aperture 805 may be an ellipse or a stadium in some embodiments. The treatment aperture 805 may have an area that is equal to about 20% to about 80% of the area of the fourth layer 605 in some examples. The treatment aperture 805 may also have an area that is equal to about 20% to about 80% of the area of a face defined by the second edge 905. A width of about 90 millimeters to about 110 millimeters and a length of about 150 millimeters to about 160 millimeters may be suitable for some embodiments of the treatment aperture 805. For example, the width of the treatment aperture 805 may be about 100 millimeters, and the length may be about 155 millimeters. In some embodiments, a suitable width for the overlay margin 915 may be about 2 millimeters to about 3 millimeters. For example, the overlay margin 915 may be coextensive with an area defined between the treatment aperture 805 and the second edge 910, and the adhesive may secure the first layer 205, the second layer 210, or both to the fourth layer 605.

Exemplary Dressing Configurations

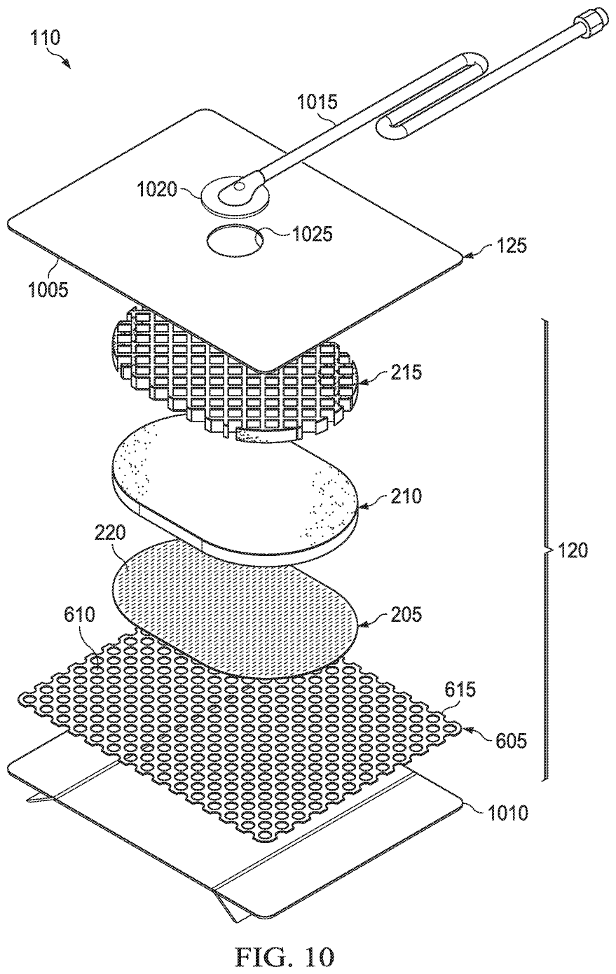

FIG. 10 is an assembly view of an example of the dressing 110 of FIG. 1, illustrating additional details that may be associated with some embodiments. The dressing 110 of FIG. 10 illustrates an example of the cover 125 with the tissue interface of FIG. 6. As illustrated in FIG. 10, the cover 125 may have larger dimensions than the first layer 205 and the second layer 210.

The dressing 110 may further include an adhesive 1005 or other type of attachment means. The adhesive 1005 may be, for example, a medically-acceptable, pressure-sensitive adhesive that extends about a periphery, a portion, or an entire surface of the cover 125. In some embodiments, for example, the adhesive 1005 may be an acrylic adhesive having a coating weight between 25-65 grams per square meter (g.s.m.). Thicker adhesives, or combinations of adhesives, may be applied in some embodiments to improve the seal and reduce leaks. In some embodiments, such a layer of the adhesive 1005 may be continuous or discontinuous. Discontinuities in the adhesive 1005 may be provided by apertures or holes (not shown) in the adhesive 1005. The apertures or holes in the adhesive 1005 may be formed after application of the adhesive 1005 or by coating the adhesive 1005 in patterns on a carrier layer, such as, for example, a side of the cover 125. Apertures or holes in the adhesive 1005 may also be sized to enhance the MVTR of the dressing 110 in some example embodiments.

As illustrated in the example of FIG. 10, in some embodiments, the dressing 110 may include a release liner 1010 to protect the adhesive 1005 prior to use. The release liner 1010 may also provide stiffness to assist with, for example, deployment of the dressing 110. The release liner 1010 may be, for example, a casting paper, a film, or polyethylene. Further, in some embodiments, the release liner 1010 may be a polyester material such as polyethylene terephthalate (PET), or similar polar semi-crystalline polymer. The use of a polar semi-crystalline polymer for the release liner 1010 may substantially preclude wrinkling or other deformation of the dressing 110. For example, the polar semi-crystalline polymer may be highly orientated and resistant to softening, swelling, or other deformation that may occur when brought into contact with components of the dressing 110, or when subjected to temperature or environmental variations, or sterilization. Further, a release agent may be disposed on a side of the release liner 1010 that is configured to contact the adhesive 1005. For example, the release agent may be a silicone coating and may have a release factor suitable to facilitate removal of the release liner 1010 by hand and without damaging or deforming the dressing 110. In some embodiments, the release agent may be a fluorocarbon or a fluorosilicone, for example. In other embodiments, the release liner 1010 may be uncoated or otherwise used without a release agent.

FIG. 10 also illustrates one example of a fluid conductor 1015 and a dressing interface 1020. As shown in the example of FIG. 10, the fluid conductor 1015 may be a flexible tube, which can be fluidly coupled on one end to the dressing interface 1020. The dressing interface 1020 may comprise an elbow connector, as shown in the example of FIG. 10, which can be placed over an aperture 1025 in the cover 125 to provide a fluid path between the fluid conductor 1015 and the tissue interface 120.

One or more of the components of the dressing 110 may additionally be treated with an antimicrobial agent in some embodiments. For example, the second layer 210 may be a foam, mesh, or non-woven coated with an antimicrobial agent. In some embodiments, the second layer 210 may comprise antimicrobial elements, such as fibers coated with an antimicrobial agent. Additionally, or alternatively, some embodiments of the first layer 205 may be a polymer coated or mixed with an antimicrobial agent. In other examples, the fluid conductor 1015 may additionally or alternatively be treated with one or more antimicrobial agents. Suitable antimicrobial agents may include, for example, metallic silver, PHMB, iodine or its complexes and mixes such as povidone iodine, copper metal compounds, chlorhexidine, or some combination of these materials.

Additionally, or alternatively, one or more of the components may be coated with a mixture that may include citric acid and collagen, which can reduce bio-films and infections. For example, the first layer 205 may be a foam coated with such a mixture.

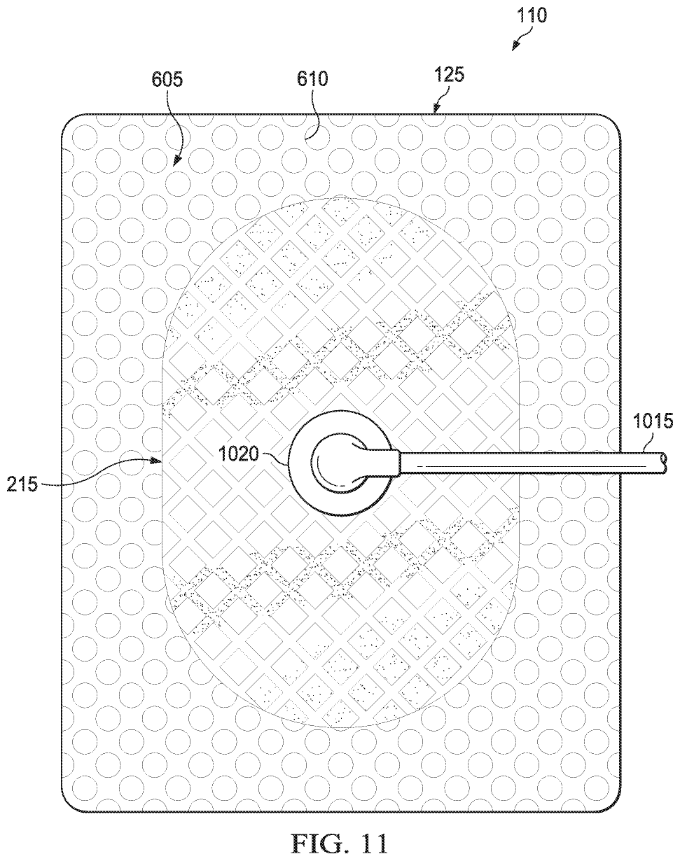

FIG. 11 is a top view of the dressing 110 in the example of FIG. 10, as assembled, illustrating additional details that may be associated with some embodiments. As illustrated in the example of FIG. 11, the cover 125 and the fourth layer 605 may have substantially the same perimeter shape and dimensions, so that the cover 125 and the fourth layer 605 are coextensive in some examples. The cover 125 may be substantially transparent, allowing visibility of the apertures 610 in some embodiments. The third layer 215 may be centrally disposed within the dressing 110. The cover 125 may be disposed over the third layer 215 and coupled to the fourth layer 605 around the third layer 215 so that at least some of the adhesive 1005 (not shown) can be disposed adjacent to the apertures 610.

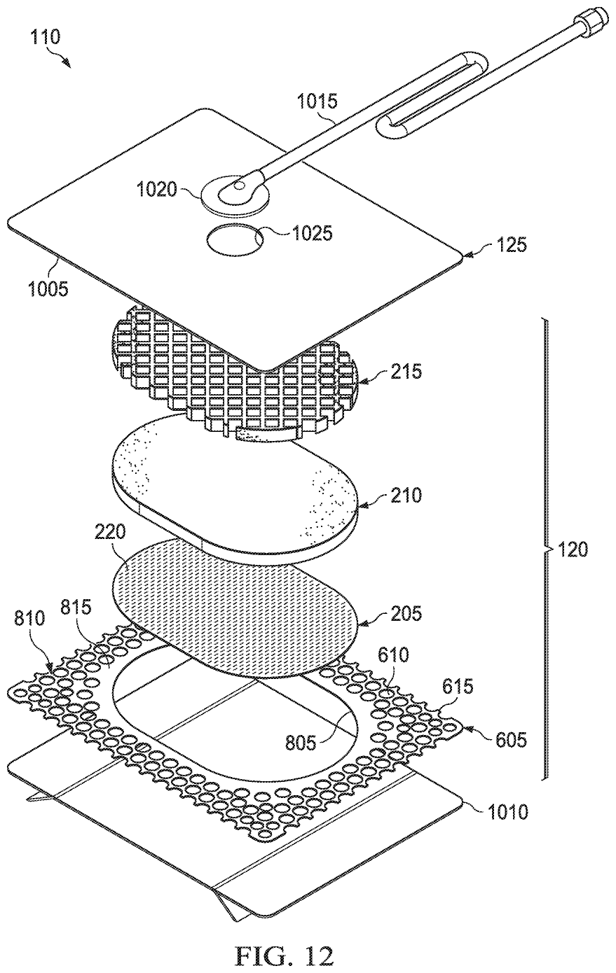

FIG. 12 is an assembly view of another example of the dressing 110 of FIG. 1, illustrating additional details that may be associated with some embodiments. The dressing 110 of FIG. 12 illustrates an example of the cover 125 with the tissue interface of FIG. 8.

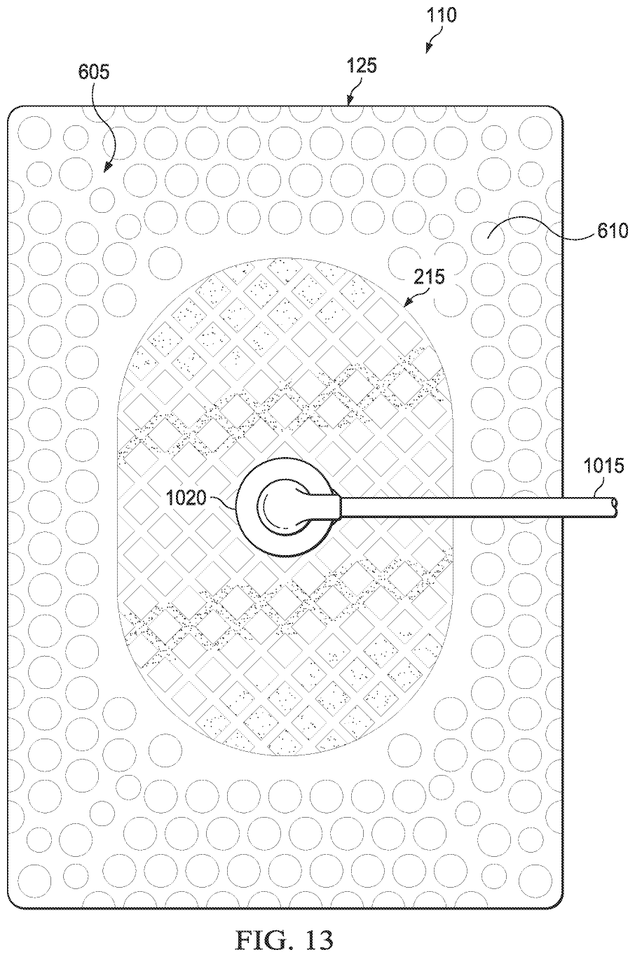

FIG. 13 is a top view of the dressing 110 of FIG. 12, illustrating additional details that may be associated with some embodiments.

Exemplary Methods of Use

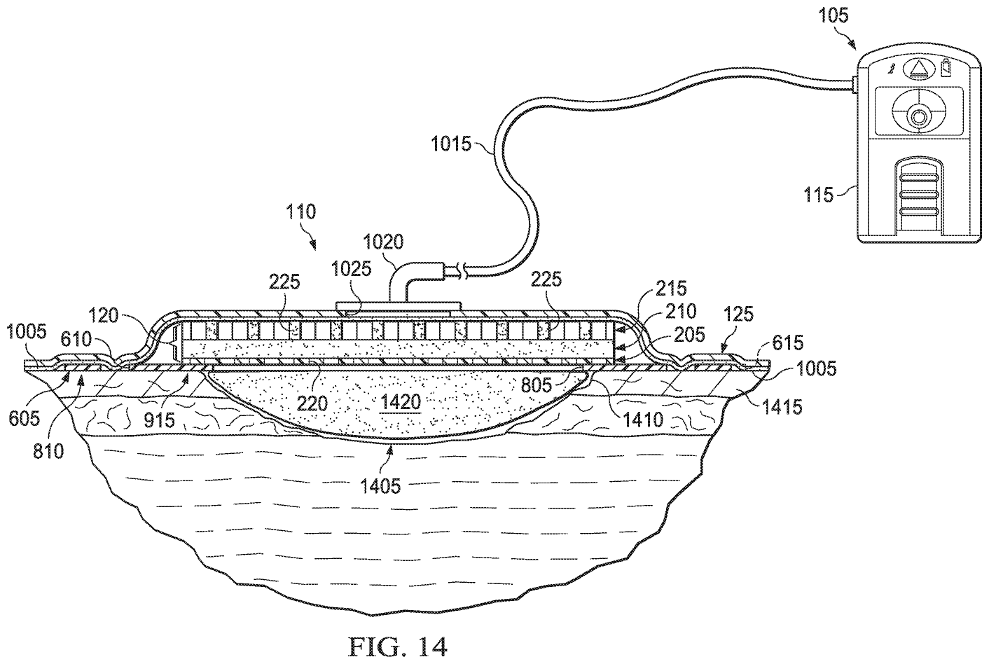

FIG. 14 is a schematic diagram of an example of the dressing 110 applied to a tissue site 1405. In the example of FIG. 14, the tissue site 1405 is a surface wound. In use, the release liner 1010 (if included) may be removed to expose the tissue interface 120, which can be placed within, over, on, or otherwise proximate to the tissue site 1405. In the example of FIG. 14, removing the release liner 1010 exposes the fourth layer 605 and a portion of the first layer 205. The first layer 205, the fourth layer 605, or both may be interposed between the second layer 210 and the tissue site 1405, which can substantially reduce or eliminate adverse interaction between the second layer 210 and the tissue site 1405. For example, the fourth layer 605 may be placed over the tissue site 1405 (including edges 1410 of the tissue site 1405) and epidermis 1415 to prevent direct contact between the second layer 205 and the tissue site 1405.

As illustrated in the example of FIG. 14, in some applications a filler 1420 may also be disposed between the tissue site 1405 and the first layer 205, the fourth layer 605, or both. For example, if the tissue site is a surface wound, a wound filler may be applied interior to the periwound, and the first layer 205 may be disposed over the filler 1420. In some embodiments, the filler 1420 may be a manifold, such as an open-cell foam. The filler 1420 may comprise or consist essentially of the same material as the second layer 210 in some embodiments. In some embodiments, the tissue interface 120 may be used as a filler. For example, the fourth layer 605 may be omitted and the first layer 205, the second layer 210, and the third layer 215 may be applied interior to the periwound area. In other examples, the first layer 205 and the fourth layer 605 may be omitted.

In some applications, the second layer 210 may provide a base manifold layer for the third layer 215 to facilitate handling and provide structural support. Additionally, or alternatively, the second layer 210, the third layer 215, or both may be cut, trimmed, or otherwise sized as appropriate in some embodiments. For example, some embodiments of the third layer 215 may have perforated sections that can be removed. Perforated sections around the periphery of the third layer 215 may be advantageous if the third layer is lightly bonded or applied in situ, so that sections over the epidermis 1415 can be removed if desired. Additionally, or alternatively, inboard sections of the third layer 215 may be removed to further increase macro-strain and contraction.

In some applications, the treatment aperture 805 may be positioned adjacent to, proximate to, or covering a tissue site. In some applications, at least some portion of the first layer 205, the fluid restrictions 220, or both may be exposed to a tissue site through the treatment aperture 805, the apertures 610, or both. The periphery 810 of the fourth layer 605 may be positioned adjacent to or proximate to tissue around or surrounding the tissue site 1405. The fourth layer 605 may be sufficiently tacky to hold the dressing 110 in position, while also allowing the dressing 110 to be removed or re-positioned without trauma to the tissue site 1405.

Removing the release liner 1010 can also expose the adhesive 1005, and the cover 125 may be attached to an attachment surface, such as the periphery 810 or other area around the treatment aperture 805 and the first layer 205. The adhesive 1005 may also be attached to the epidermis 1415 peripheral to the tissue site 1405, around the first layer 205, the second layer 210, and the third layer 215. For example, the adhesive 1005 may be in contact with the epidermis 1415 through the apertures 610 in at least the periphery 810 of the fourth layer 605. The adhesive 1005 may also be in contact with the epidermis 1415 around the edges 615.

Once the dressing 110 is in a desired position, the adhesive 1005 may be pressed through the apertures 610 to bond the dressing 110 to the attachment surface. The apertures 610 at the edges 615 may permit the adhesive 1005 to flow around the edges 615 for enhancing the adhesion of the edges 615 to an attachment surface.

In some embodiments, the apertures 610 may be sized to control the amount of the adhesive 1005 exposed through the apertures 610. For a given geometry of the fourth layer 605, the relative sizes of the apertures 610 may be configured to maximize the surface area of the adhesive 1005 exposed through the apertures 610 at corners of the fourth layer 605. In some embodiments, the corners be rounded to have a radius of about 10 millimeters. Further, in some embodiments, three of the apertures 610 may be positioned in a triangular configuration at the corners to maximize the exposed surface area for the adhesive 1005. In other embodiments, the size and number of the apertures 610 in the corners may be adjusted as necessary, depending on the chosen geometry of the corners, to maximize the exposed surface area of the adhesive 1005.

In some embodiments, the bond strength of the adhesive 1005 may vary based on the configuration of the fourth layer 605. For example, the bond strength may vary based on the size of the apertures 610. In some examples, the bond strength may be inversely proportional to the size of the apertures 610. Additionally or alternatively, the bond strength may vary in different locations, for example, if the size of the apertures 610 varies. For example, a lower bond strength in combination with larger apertures may provide a bond comparable to a higher bond strength in locations having smaller apertures.

The geometry and dimensions of the tissue interface 120, the cover 125, or both may vary to suit a particular application or anatomy. For example, the geometry or dimensions of the tissue interface 120 and the cover 125 may be adapted to provide an effective and reliable seal against challenging anatomical surfaces, such as an elbow or heel, at and around a tissue site. Additionally or alternatively, the dimensions may be modified to increase the surface area for the fourth layer 605 to enhance the movement and proliferation of epithelial cells at a tissue site and reduce the likelihood of granulation tissue in-growth.

Further, the dressing 110 may permit re-application or re-positioning, to correct air leaks caused by creases and other discontinuities in the dressing 110, for example. The ability to rectify leaks may increase the efficacy of the therapy and reduce power consumption in some embodiments.

If not already configured, the dressing interface 1020 may be disposed over the aperture 1025 and attached to the cover 125. The fluid conductor 1015 may be fluidly coupled to the dressing interface 1020 and to the negative-pressure source 105.

In the example of FIG. 14, the treatment aperture 805 can provide an open area in the fourth layer 605 for delivery of negative pressure and passage of exudate and other types of fluid through the first layer 205, the second layer 210, and the third layer 215. In other examples, the apertures 610 may provide a suitable open area. In yet other examples, the fourth layer 605 may be omitted.

Negative pressure applied through the tissue interface 120 can also create a negative pressure differential across the fluid restrictions 220 in the first layer 205, which can open or expand the fluid restrictions 220. For example, in some embodiments in which the fluid restrictions 220 may comprise substantially closed fenestrations through the first layer 205, a pressure gradient across the fenestrations can strain the adjacent material of the first layer 205 and increase the dimensions of the fenestrations to allow liquid movement through them, similar to the operation of a duckbill valve. Opening the fluid restrictions 220 can allow exudate and other liquid movement through the fluid restrictions 220 into the second layer 210. The second layer 210 and the third layer 215 can provide passage of negative pressure and exudate, which can be collected in the container 115.

Changes in pressure can also cause the second layer 210 and the third layer 215 to expand and contract. Negative pressure can also cause the holes 225 to collapse, allowing further contraction of the third layer 215. Further contraction of the third layer 215 can be transferred as closure forces to the edges 1410 of the tissue site 1405. In some embodiments, the holes 225 may be configured to cause contraction of the third layer 215 before contraction of the second layer 210, which can allow the second layer 210 to provide structural integrity to the third layer 215 without substantially impacting or reducing overall closure forces from the third layer 215. For example, the second layer 210 may be sufficiently stiff to contract only at negative pressure of at least 60-70 mmHg, and the holes 225 may allow the third layer 215 to contract under negative pressure of 50 mmHg or less. In some embodiments, the density of the second layer 210 and the third layer 215 may be configured to provide differential collapse characteristics. For example, a suitable ratio of the density of the second layer 210 to the density of the third layer 215 may be in a range of about 2.5 to about 3.3 in some embodiments.

The first layer 205, the fourth layer 605, or both may protect the epidermis 1415 from irritation that could be caused by expansion, contraction, or other movement of the second layer 210. For example, in some embodiments, the overlay margin 915 may be disposed between the second layer 210 and the epidermis 1415. The first layer 205 and the fourth layer 605 can also substantially reduce or prevent exposure of a tissue site to the second layer 210, which can inhibit growth of tissue into the second layer 210. For example, the first layer 205 may cover the treatment aperture 810 to prevent direct contact between the second layer 210 and a tissue site.

If the negative-pressure source 105 is removed or turned off, the pressure differential across the fluid restrictions 220 can dissipate, allowing the fluid restrictions 220 to close and prevent exudate or other liquid from returning to the tissue site 1405 through the first layer 205.

Additionally, or alternatively, instillation solution or other fluid may be distributed to the dressing 110, which can increase the pressure in the tissue interface 120. The increased pressure in the tissue interface 120 can create a positive pressure differential across the fluid restrictions 220 in the first layer 205, which can open the fluid restrictions 220 to allow the instillation solution or other fluid to be distributed to the tissue site 1405.

The systems, apparatuses, and methods described herein may provide significant advantages. For example, some dressings for negative-pressure therapy can require time and skill to be properly sized and applied to achieve a good fit and seal. In contrast, some embodiments of the dressing 110 provide a negative-pressure dressing that is simple to apply, reducing the time to apply and remove. In some embodiments, for example, the dressing 110 may be a fully-integrated negative-pressure therapy dressing that can be applied to a tissue site (including on the periwound) in one step, without being cut to size, while still providing or improving many benefits of other negative-pressure therapy dressings that require sizing. Such benefits may include good manifolding, beneficial granulation, protection of the peripheral tissue from maceration, protection of the tissue site from shedding materials, and a low-trauma and high-seal bond. These characteristics may be particularly advantageous for surface wounds having moderate depth and medium-to-high levels of exudate. Some embodiments of the dressing 110 may remain on the tissue site for at least 5 days, and some embodiments may remain for at least 7 days.

Antimicrobial agents in the dressing 110 may extend the usable life of the dressing 110 by reducing or eliminating infection risks that may be associated with extended use, particularly use with infected or highly exuding wounds.

Additionally, or alternatively, the tissue interface 120 can provide a manifold structure that can also provide radial closure forces under negative pressure, and further can substantially reduce or prevent tissue growth into the manifold structure and consequent trauma on removal. The tissue interface 120 may be particularly advantageous for deep and complex wounds where there may have been significant debridement of tissue and an opening that needs to be closed. Some embodiments of the tissue interface 120 can reduce overall wound size and area by laterally and uniformly collapsing under negative pressure. The tissue interface 120 may also provide a consistent surface topology to the wound bed, which can improve cosmetic outcomes. Further, the third layer 215 may be removed after the edges 1410 have been sufficiently drawn together and edema reduced.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or rearranged in various configurations for purposes of sale, manufacture, assembly, or use. In some configurations, layers of the tissue interface 120 may be rearranged. For example, the third layer 215 may be disposed between the first layer 205 and the second layer 210. Additionally, or alternatively, the first layer 205 may be removed in some examples.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A dressing for treating a tissue site with negative pressure, the dressing comprising:
a fluid control layer comprising a plurality of fluid restrictions;
a first manifold layer adjacent to the fluid restrictions, the first manifold layer having a first density; and
a second manifold layer having perforations adjacent to the first manifold layer, the second manifold layer having a second density that is less than the first density.

2. The dressing of claim 1, wherein a ratio of the first density to the second density is in a range of about 2.5 to about 3.3.

3. The dressing of claim 1, wherein the first density is about 0.65 grams per cubic centimeter, and the second density is in a range of about 0.20 to about 0.26 grams per cubic centimeter.

4. The dressing of claim 1, wherein the perforations define an open area in the second manifold layer of about 30% to about 70%.

5. The dressing of claim 1, wherein the perforations are open right cylinders.

6. The dressing of claim 1, wherein the perforations are open right cylinders having a right section that is a polygon.

7. The dressing of claim 1, wherein the perforations are open right cylinders having a right section that is a regular polygon.

8. The dressing of claim 1, wherein the perforations are open right cylinders having a square right section.

9. The dressing of claim 1, wherein the perforations are arranged in a uniform pattern.

10. The dressing of claim 1, wherein the perforations are arranged in a pattern of rows.

11. The dressing of claim 1, wherein:
the first manifold layer is comprised of foam having a thickness in a range of about 1 millimeter to about 6 millimeters; and
the second manifold layer is comprised of foam having a thickness in a range of about 6 millimeters to about 20 millimeters.

12. The dressing of claim 1, wherein:
the first manifold layer is comprised of felted foam having a thickness in a range of about 1 millimeter to about 3 millimeters; and
the second manifold layer is comprised of foam having a thickness in a range of about 10 millimeters to about 20 millimeters.

13. The dressing of claim 1, wherein the fluid control layer comprises a film of polyurethane.

14. The dressing of claim 13, wherein the fluid restrictions comprise slits in the film.

15. The dressing of claim 14, wherein the slits each have a length in a range of about 2 millimeters to about 5 millimeters.

16. The dressing of claim 14, wherein the slits each have a length of about 3 millimeters.

17. A dressing for treating a tissue site with negative pressure, the dressing comprising:
a first layer comprising a fluid control layer having a plurality of fluid restrictions;
a second layer comprising a base manifold adjacent to the fluid restrictions, the base manifold configured to deform laterally at a first negative pressure; and
a third layer comprising a closure manifold adjacent to the base manifold, the closure manifold configured to deform laterally at a second negative pressure that is less than the first negative pressure.

18. The dressing of claim 17, wherein:
the first negative pressure is at least 60 mmHg; and
the second negative pressure is less than 50 mmHg.

19. The dressing of claim 17, wherein the closure manifold comprises a plurality of holes.

20. The dressing of claim 17, wherein the closure manifold comprises a plurality of through-holes.

21. The dressing of claim 17, wherein the closure manifold comprises a plurality of through-holes and a web of struts separating the through-holes, the struts having a substantially uniform thickness.

22. The dressing of claim 17, wherein:
the base manifold comprises open-cell foam having a thickness in a range of about 1 millimeter to about 6 millimeters; and
the closure manifold comprises foam having a thickness in a range of about 6 millimeters to about 20 millimeters.

23. The dressing of claim 17, wherein:
the base manifold comprises felted open-cell foam having a thickness in a range of about 1 millimeter to about 3 millimeters; and
the closure manifold comprises open-cell foam having a thickness in a range of about 10 millimeters to about 20 millimeters.

24. The dressing of claim 17, wherein the fluid control layer comprises a film of polyurethane.

25. (canceled)

26. A method for promoting closure of a tissue site with negative pressure, the method comprising:
applying the dressing of claim 1 or claim 17 to the tissue site;
attaching a cover to an attachment surface around the tissue site to seal the dressing over the tissue site;
fluidly coupling the dressing to a negative-pressure source; and
applying negative pressure from the negative pressure source to the dressing.

27. (canceled)
